(19) United States
(12) Patent Application Publication
(10) Pub. No.: US 2013/0230904 A1
(43) Pub. Date: Sep. 5, 2013

(54) LENSED AND STRIPED FLAT PANEL PHOTOBIOREACTORS

(71) Applicants: RONALD SURYO, Fairfax, VA (US); JOHN T. WYATT, Alexandria, VA (US)

(72) Inventors: RONALD SURYO, Fairfax, VA (US); JOHN T. WYATT, Alexandria, VA (US)

(73) Assignee: EXXONMOBIL RESEARCH AND ENGINEERING COMPANY, Annandale, NJ (US)

(21) Appl. No.: 13/599,138

(22) Filed: Aug. 30, 2012

Related U.S. Application Data
(60) Provisional application No. 61/529,522, filed on Aug. 31, 2011.

(51) Int. Cl. C12M 1/00

(52) U.S. Cl. CPC C12M 21/02
USPC 435/257.1; 435/292.1

(57) ABSTRACT

The surface or surfaces of the walls of a closed photobioreactor are modified to generate light and dark regions. For example, the interior and/or exterior surfaces of the walls of the photobioreactor's chamber can be modified such that they form or incorporate convex and/or concave lenses. The concavity and/or convexity of the lenses redirect incident light rays so that the light is focused to specific points within the chamber, creating multiple light and dark regions within the photobioreactor. Alternatively, or in addition, a pattern of opaque material can be utilized on the walls of the chamber to generate light and dark regions. The number, location and shape of the light and dark regions are controlled by the design of the concavities and/or convexities and/or opaque patterns incorporated into the various surfaces of the photobioreactor.

### CROSS-REFERENCE TO RELATED APPLICATIONS

This application claims the benefit of U.S. Ser. No. 61/529,522, filed Aug. 31, 2011, the entire disclosure of which is hereby incorporated by reference herein.

### FIELD OF THE INVENTION

The present invention relates to photobioreactors for growing photosynthetic micro-organisms. More particularly, the present invention relates to flat panel photobioreactors that utilize lenses and/or an opaque material to induce an effective light/dark cycle.

### BACKGROUND OF THE INVENTION

Microalgae and cyanobacteria (hereinafter “microalgae”) primarily require simple mineral nutrients and carbon dioxide (CO₂) for growth and reproduction. With over 40,000 identified species, microalgae represent a very diverse group of organisms. Through photosynthesis, microalgae convert water and CO₂ into bioproducts. Examples of microalgae bioproducts include biofuels, pigments, proteins, fatty acids, and carbohydrates, to name a few.

The use of microalgae to produce renewable biofuels and other high-value products is scientifically and environmentally sound—but the economic viability of such operations is largely limited by the efficiency and cost-effectiveness of the industrial-scale vessels in which the microalgae is grown. Although hybrid systems exist, most growth systems can be categorized into one of two types. The first type of growth system is an open pond—most often a raceway pond although other types of ponds, such as circular ponds, have been employed. The second type of growth system is a closed photobioreactor (alternatively referred to herein as a “photobioreactor” or “PBR”).

Raceway ponds are typically shallow ponds, about 30 cm deep, constructed as a continuous loop around which water containing microalgae (“algae culture”) is circulated mechanically via a paddle wheel. These and other open ponds have the advantage of being relatively simple and cheap in construction and maintenance.

Open ponds, however, have a number of disadvantages. First, open ponds do not utilize light efficiently. Algae absorb light and the depth of the microalgae culture required for effective circulation is generally deeper than incident light can penetrate. Second, the mixing in open ponds dissipates rapidly as microalgae culture moves away from the paddlewheel and essentially becomes plug flow throughout the majority of the pond. Third, open ponds are subject to environmental factors such as wind, rain, temperature, and evaporation. Fourth, open ponds are prone to contamination by airborne micro-organisms and dust that lower productivity even further and sometimes cause culture failure. The poor light utilization, poor mixing, limited environmental control and contamination issues result in relatively low biomass and bioproduct productivity. Typical biomass productivity for an open pond is on the order of 10-20 g/m²/day of dry microalgae. These significant productivity drawbacks prompted the development of closed photobioreactors.

Closed photobioreactors are typically constructed of transparent tubes or containers in which microalgae culture is mixed by either a pump or injected gas (e.g., air and/or CO₂). Multiple types of closed photobioreactor designs have been proposed and developed.

One type of closed photobioreactor is a tubular photobioreactor. Tubular photobioreactors generally comprise serpentine or helical tubing made of glass, acrylic or other plastic and a gas exchange vessel wherein CO₂ and air are added and oxygen (O₂) is removed. The gas exchange vessel is typically connected to the ends of the tubing. Recirculation of the microalgae culture between the gas exchange vessel and the tubing is generally performed by a pump or an air-lift. In tubular photobioreactors, incident light is generally able to enter the tubing from multiple directions. Further, the tubing diameter is typically thinner than the depth of an open pond. Therefore, incident light is utilized more effectively. In addition, mixing and environmental control is easier to accomplish in tubular photobioreactors. As a result, tubular photobioreactors generally enable an increase in algal biomass and bioproduct productivity.

But tubular photobioreactors also have a number of disadvantages. First, tubular photobioreactors tend to have large “dark zones” or “dark volumes” associated with the gas exchange vessel where dissolved oxygen is exchanged with carbon dioxide. Algal cells entering such dark zones cannot perform photosynthesis and, therefore, consume cell mass through cellular respiration. Second, tubular photobioreactors tend to retain and accumulate high concentrations of molecular oxygen evolved from photosynthesis—because there is nowhere for oxygen to go until it reaches the gas exchange vessel. High concentrations of oxygen inhibit photosynthesis and thus biomass and bioproduct productivity. Third, the mechanical pumps often employed in tubular photobioreactors to facilitate culture mixing and circulation are, by necessity, quite powerful, and can cause cell damage. Due to the hydrodynamic stress created by the mechanical pumps, only a limited number of robust algal species are able to thrive in a tubular photobioreactor.

Another type of closed photobioreactor is a flat panel or flat plate photobioreactor. Flat panel photobioreactors comprise a large chamber for holding fluid. The width of the chamber is generally thinner than its length and height—a typical chamber being 1 to 3 m long, 0.5 to 2 m high and 3 to 5 cm wide. Flat panel photobioreactors typically exhibit all the benefits of tubular photobioreactors without the detriments. In addition, when an array of flat panel photobioreactors are employed, one in front of the other, incident light bounces between the devices and becomes diffuse. This, in turn, increases algal biomass and product productivity.

FIG. 1 illustrates a typical flat panel PBR. In FIG. 1, a flat panel PBR 100 comprises a rectangular chamber 1 formed by multiple walls (i.e., “panels”) consisting of front wall 2A, back wall 2B, top wall 3A, opposing side walls 3B and 3C and bottom wall 3D. The walls of the chamber are transparent or translucent to allow light to reach the microalgae culture. In a typical flat panel PBR, as the name implies, the walls are typically flat and smooth on the inner and outer surface. During operation, chamber 1 is substantially filled with an aqueous media 5 comprising a culture of microalgae. Media 5 is drawn from a pipe 8 that draws from a media holding tank, a header or another PBR. The position where pipe 8 connects to chamber 1 is not particularly important. In practice, chamber 1 is not fully filled with media 5. Instead, a head space 6 is maintained at the top of chamber 1 to provide a region for gas exchange. Nutrient gases (e.g., CO₂, air, or flue gas) required for growing microalgae are then injected into chamber 1. Gas injection is accomplished by way of a sparger 11—a pipe that runs horizontally along the lower interior of chamber 1 having multiple outlets (not shown) that distribute nutrient gas in the form of gas bubbles 12. Unused gases (e.g., CO₂ and air) and photosynthetic byproduct gases (e.g., O₂) accumulate in head space 6 and are released into the atmosphere through a relatively high opening 10 in a wall (e.g., in side wall 3C). If an array of flat panel PBRs are employed, then media 5 may move out of chamber 1 into the chamber of another flat panel PBR (not shown) through pipe 9. The exact location of pipe 9 can vary. Additionally, chamber 1 may contain one or more baffles. In this case, four baffles (each 13) are shown. Baffles are a known means to enhance mixing.

It is known that continuous fluctuation of light enhances microalgae biomass and product productivity—a mechanism known as the “intermittent light effect” or “light/dark cycle”. Too much light at any given time causes photo-inhibition in microalgae. In addition, constant high light causes photo-acclimation in microalgae - thereby reducing its ability to process lower intensity light. By cycling microalgae in and out of the light, higher photosynthetic efficiency can be maintained and denser and more productive colonies can be grown. Studies show that exposing microalgae to continuous light/dark cycles enhances overall growth productivity anywhere from 20% to 100%.

It is also known that an extremely rapid alteration between high light intensity light and darkness enhances photosynthetic efficiency in microalgae even further. Due to the high light-harvesting efficiency of chlorophyll in microalgae, algae absorb all the light that reaches them even though they cannot use all the photons. The energy absorbed from the unused photons is released as heat. By cycling algae between light/dark periods of approximately 1-10 ms, depending on the particular type of algae, photo-efficiency is maximized. This is called the “flashing light effect.”

Unfortunately, inducing light/dark cycle in a closed photobioreactor is costly. One method for inducing light/dark cycle is vigorous mixing. Mixing causes microalgae to move in and out of light regions at the surface of the photobioreactor and darker regions in the interior. But mixing consumes energy and, thereby, cost. For low margin products such as biofuel—the mixing required can be cost prohibitive. Another method for inducing light/dark cycle is to utilize a flashing synthetic light. However, this is even more energy intensive and cost prohibitive.

It would be desirable to increase microalgae biomass and bioproduct productivity without significant cost. It would be desirable to induce light/dark cycle and/or the flashing light effect in a photobioreactor in a relatively passive manner This would better enable commercialization of algae derived bioproducts such as biofuels.

### SUMMARY OF THE INVENTION

The invention pertains to closed photobioreactors for growing photosynthetic micro-organisms comprising a chamber for holding media. The chamber is formed from a plurality of panels. At least one panel contains translucent or transparent portions and, further, redirects or absorbs light (preferably incident sunlight) to provide a plurality of alternating light and dark regions within the chamber.

The requisite light and dark regions may be made by lenses. More particularly, the chamber of a photobioreactor may be made of one or more translucent or transparent panels that form or comprise one or more convex and/or concave lenses. The lenses converge and/or diverge light to generate a plurality of alternating light and dark regions within the chamber. Preferably, the chamber panels form or comprise multiple convex and/or concave lenses. Any lenses that converge or diverge light are suitable for use in the invention including, but not limited to, lenses selected from the types consisting of biconvex, biconcave, plano-convex, plano-concave and meniscus (i.e., convex-concave) lenses and any combination thereof. The lenses focus some of the incident light rays striking the chamber toward specific points within the chamber and away from other points within the chamber, thereby generating multiple light and dark regions.

Alternatively or additionally to making light and dark regions by means of lenses, the requisite light and dark regions may be made using a pattern of opaque material (typically, horizontal stripes of opaque film). More particularly, the chamber of a photobioreactor comprises a pattern of alternating transparent or translucent regions and opaque regions to generate a plurality of light and dark regions within the chamber. Relatively lighter regions will be created adjacent to the transparent or translucent regions and relatively darker regions will be created adjacent to the opaque regions. Preferably, the opaque regions are also reflective.

The invention also pertains to a method for increasing the biomass and/or bioproduct productivity of photosynthetic micro-organisms (preferably microalgae) in a photobioreactor comprising at least two steps. The first step is redirecting or blocking at least a portion of the light that reaches a surface of the photobioreactor to create a plurality of alternating light and dark regions within the photobioreactor. The second step is moving micro-organisms directionally through said light and dark regions. The step of creating a plurality of light and dark regions may be achieved using a photobioreactor having one or more features of the photobioreactor described above.

The invention also pertains to a method for generating an alternating vertical current in a closed photobioreactors comprising a chamber for growing photosynthetic micro-organisms. The method comprises the steps of positioning one or more baffles in the chamber and releasing a gas (e.g., nutrient gas) into the bottom of the chamber at different rates on either side of each baffle. The photobioreactor in accordance with this aspect of the invention may have one or more features of photobioreactor as described (i.e., having a plurality of light and dark regions) or alternatively, the photobioreactor may lack such features.

The photobioreactors and methods described herein provide a relatively passive means for exposing photosynthetic micro-organisms to a regularly alternating light/dark cycle. In the invention, as the micro-organisms move, or are made to move, directionally through the photobioreactors, they experience alternating regions of relatively high and low light without significant energy input. When lenses are employed, the photobioreactors and methods described herein also focus light more intently so that it penetrates deeper into colonies of light absorbing micro-organisms. This, in turn, permits the utilization of significantly wider chambers, thereby reducing the total number of chambers required to achieve a given productivity and reducing capital and operational costs. Alternatively, when reflective materials are employed, the photobioreactors and methods described herein also reduce the heating experienced by the micro-organisms in the chamber and, thereby, reduce the need and cost for cooling.

### BRIEF DESCRIPTION OF THE DRAWINGS

The following figures are for illustrative purposes only and are not intended to limit the scope of the present invention in any way:

FIG. 1 is an illustration of a typical flat panel PBR.

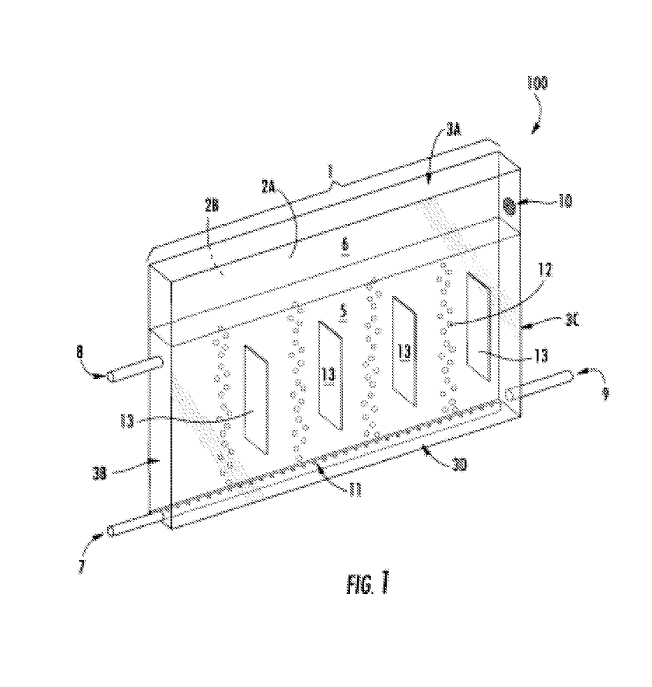

FIG. 2 is a cross-sectional side view of the chamber in one embodiment of a lensed flat panel PBR constructed in accordance with the present invention.

FIG. 3 is a perspective view of one wall of FIG. 2.

FIG. 4 is a cross-sectional side view of the chamber in a second embodiment of a lensed flat panel PBR constructed in accordance with the present invention.

FIG. 5 is a perspective view of one wall of FIG. 4.

FIG. 6 is a cross-sectional side view of the chamber in a third embodiment of a lensed flat panel PBR constructed in accordance with the present invention.

FIG. 7 is an illustration of a striped flat panel PBR constructed in accordance with the present invention.

FIG. 8 illustrates a sparger/baffle system for a flat panel PBR constructed in accordance with the present invention.

### DETAILED DESCRIPTION OF THE EMBODIMENTS

### Introduction

The invention pertains to photobioreactors for growing photosynthetic micro-organisms and a method of growing photosynthetic micro-organisms (preferably, microalgae). The invention is most often explained in terms of a flat panel photobioreactor and preferably is a flat panel photobioreactor. However, the invention is not so limited. Accordingly, other types of photobioreactors may be employed in accordance with the invention as long as the micro-organisms move along a roughly discernible directional path.

In general, in the photobioreactors of the current invention, one or more walls of a growth chamber are formed or altered so that incident light (preferably incident sunlight) hitting the chamber's walls reaches some regions of chamber in a greater degree than other regions, thereby creating a multitude of alternating light and dark regions within the chamber. As the photosynthetic micro-organisms flow directionally through the chamber, the micro-organisms experience regions of high light and low light—thereby experiencing the light/dark cycle without the need of extensive, and energy intensive, mixing.

For the purpose of the invention, “light/dark profile” means the distribution of light regions and dark regions throughout the chamber of the photobioreactor. More particularly a “vertical light/dark profile” refers to the manner in which the light and dark regions are distributed along the height of the chamber and a “horizontal light/dark profile” refers to the manner in which the light and dark regions are distributed along the length of the chamber. In this regard, “dark regions” are regions that preferably have little or no light and at least have 50% or less light than the “light regions” within the chamber.

The multiple alternating light and dark regions may be created by using one or more translucent or transparent chamber walls that form, or otherwise contain, one or more concave and/or convex lenses. Alternatively or additionally, the multiple alternating light and dark regions are created by using one or more translucent or transparent chamber walls that form or are treated to otherwise contain opaque regions (e.g., by application of spaced stripes of a reflective film). The invention also provides methods of growing photosynthetic micro-organisms, enabled by the photobioreactors disclosed. Methods are also disclosed that employ new baffle and sparger configurations to create alternating currents to maximize growth. These and other embodiments are explained in further detail below.

### Lensed PBR

The photobioreactors of the invention, may comprise one or more surfaces of one or more walls of a chamber incorporating one or more (and preferably multiple) concavities and/or convexities to create lenses. These lenses alter the direction of incident light flowing to the chamber to generate regions of relatively high and relatively low light in the chamber. The lenses may be, for example, selected from the group consisting of biconvex, biconcave, plano-convex, plano-concave and meniscus (i.e., convex-concave) lenses, and any combination thereof, so long as the lenses converge or diverge light in a substantially predictable manner. For the purposes of this description, this first arrangement will be called a “lensed PBR.”

The chamber walls of a lensed PBR are typically made of smooth rigid glass, acrylic or other plastic. The lenses may be adhered to, or formed into, the chamber walls. Preferably, from a structural integrity standpoint, the lenses are formed one or more walls during manufacture by blow molding, form molding, etching and/or other conventional manufacturing techniques.

Any chamber wall containing lenses may contain lenses only on the interior surface, only on the exterior surface, or on both the interior and exterior surfaces.

Preferably, the lenses are positioned on at least the interior surface of a chamber wall since the curvature of each lens provides disturbances to the media flow and, thereby, more turbulent mixing. However, in certain designs, fouling may be a problem that forces the lenses to be located only on the outside surface. Further, in arrangements using bi-convex, bi-concave or meniscus lenses, forming one curvature of each lens on the outside of a chamber wall and the other on the inside of the chamber wall is a convenient method for making the two required curvatures.

Any of the chamber walls may incorporate lenses. Preferably, the lenses are located on the two opposing chamber walls with the most surface area in order to provide regularly dispersed light and dark regions throughout the chamber. Preferably, but not necessarily, the lenses on the opposite walls directly align with one another. The exact configuration of the lenses depends on the specific light/dark profile desired. This, in turn, depends on the type of photobioreactor utilized (e.g., tubular or flat panel) and the optimal light/dark profile requirement of the particular species of micro-organism being grown.

Preferably, the lensed PBR is a flat panel photobioreactor (hereinafter “lensed flat panel PBR”). Even more preferably, the lensed flat panel PBR has a chamber comprising multiple lenses in or on its front and/or back panels (i.e., the panels having the largest surface area). The lenses are positioned in parallel rows to create a stacked series of alternating rows of light exposure within the chamber, wherein rows of light regions are interspaced with rows of dark regions, each extending parallel to the chambers horizontal axis (i.e., length). Thus, light is substantially uniform across a majority, if not all, horizontal cross-sections taken parallel to the length of the chamber but alternates along a majority, if not all, vertical cross-sections taken parallel to the height of the chamber. This creates a vertical light/dark profile that consistently alternates between light and dark across the majority, and preferably the entire, vertical axis (height) of the photobioreactor and a horizontal light/dark profile that remains substantially uniform (i.e., stays consistently light or consistently dark) across the majority, and preferably the entire, horizontal length of the photobioreactor.

In a lensed flat panel PBR, a vertically alternating light/dark profile is desired since micro-organisms typically flow up and down in the chamber roughly in-line with vertical currents created by one or more spargers at the bottom of the chamber. Flow within a lensed flat panel PBR is typically generated by bubbling CO₂, air, and/or flue gas through one or more spargers located at the bottom of the flat panel. As the bubbles rise, they generate liquid flow circulation within the chamber causing the micro-organisms in the media to rise and fall.

In a lensed flat panel PBR, the frequency of the light/dark cycle can be optimized by varying the interplay between the curvature of the lenses and the bubbling rate of resource gases (e.g., air and CO₂). The bubbling rate changes the velocity of the fluid which, in turn, changes the frequency of the light/dark cycle experienced by the micro-organisms. Preferred configurations, therefore, include a lensed flat panel PBR wherein the interplay between the radius of the curvature of the lenses and the bubbling rate produced by one or more spargers at or near the bottom of the chamber combine to expose micro-organisms in the chamber to a light/dark cycle that alternates at a frequency ranging from 25 microseconds to 60 seconds. Preferably, the light/dark cycle alternates at a frequency ranging from 1 millisecond to 60 seconds. Ideally, the alternating light/dark cycle alternates at a frequency ranging from 1 second to 10 seconds.

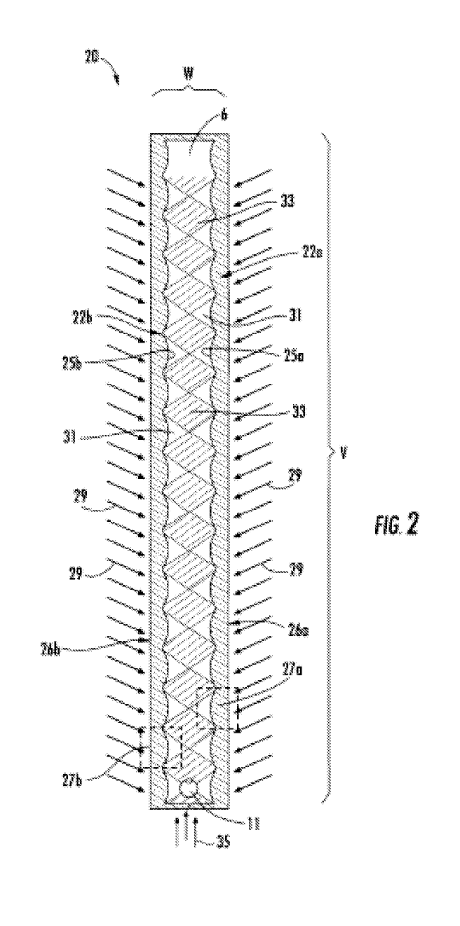

One example of a lensed flat panel PBR is illustrated by FIG. 2. FIG. 2 provides a cross sectional side view a chamber 20 of a lensed flat panel PBR.

In FIG. 2, chamber 20 has two front and back walls 22a and 22b, respectively. Front and back walls 22a and 22b can be made of any smooth, rigid, transparent material such as glass or plastic, for example acrylic. Chamber 20 also has top and bottom walls (not labeled) and side walls (not shown). The chamber has a width W and a vertical height V. The cross-sectional perspective is looking down the length (not shown) of chamber 20.

In FIG. 2, the exterior surface 26a and 26b of each wall 22a and 22b, respectively, is flat. In contrast the interior surface 25a and 25b of walls 22a and 22b, respectively, are curved. Together the interior and exterior surfaces of front and back walls 22a and 22b, respectively, form a series of opposing plano-convex lenses 27a and 27b wherein the curvature of each plano-convex lens points inward towards the intervening media (not shown) in chamber 20. In other words, each convex lens on front wall 22a faces across the intervening media toward an opposing convex lens 27b on opposing back wall 22b of the chamber 20. Incident light 29 hits the exterior surface 26a and 26b of walls 22a and 22b, respectively, and is then focused to a focal point located inside chamber 20.

In FIG. 2, the direction of incident light 29 is shown by arrows and hits chamber 20 at an angle. However, the direction of incident light 29 is for illustration purposes only and need not hit chamber 20 at an angle, much less at any particular angle, to produce the desired effect.

In FIG. 2, the curvature of lenses 27a and 27b is shaped to focus light into chamber 20 to a focal point approximately half way between the interior surfaces of walls 22a and 22b. The result is that even though incident light 27 hits the entire exterior surface 26a and 26b of walls 22a and 22b, respectively, triangular regions of high light 31 and diamond shaped regions of low light (i.e., dark regions) 33 are created inside chamber 20. Because lenses 27a and 27b are distributed one after the other along the entire height of the front and back walls 22a and 22b, high light regions 31 and dark regions 33 are created throughout the entire vertical profile V of chamber 20. It should be noted that the exact shape of the light and dark regions may change based on the angle of the incoming light. Further, if the photobioreactor does not reposition itself to continuously face the sun, then the exact shape of the pattern of light and dark regions may vary as the angle of light from the sun throughout the day.

In FIG. 2, media (not labeled) fills the chamber up to head space 6. The direction of the media flow is indicated by upward arrows 35. Sparger 11 located near the bottom of chamber 20 generates current by introducing bubbles of gas (not shown) into the chamber. The flowing current causes the media, and therefore the micro-organisms therein, to move vertically in chamber 20 through alternating high light and dark regions.

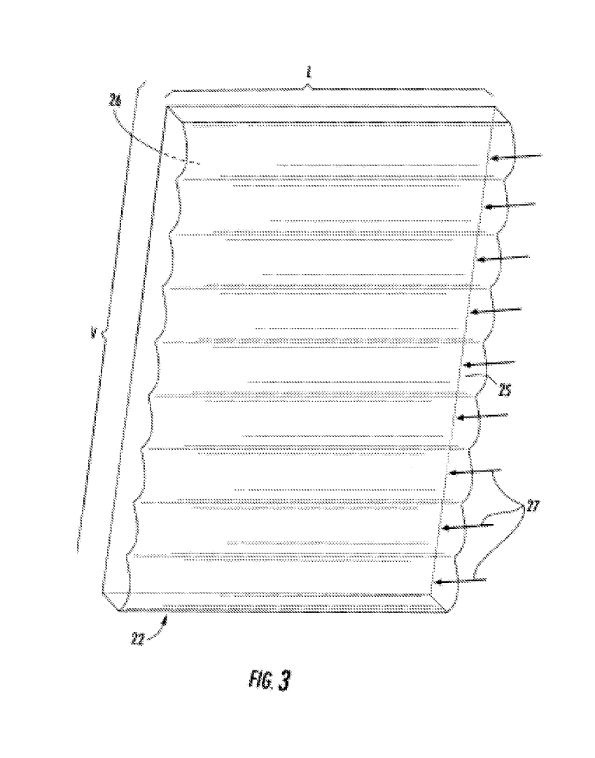

FIG. 3 further illustrates the lensed flat panel PBR embodiment shown in FIG. 2. More particularly, FIG. 3 shows a perspective view of one wall 22 in chamber 20 of FIG. 2. In FIG. 3, wall 22 has a length L and a height V. The outer surface 26 is substantially smooth and flat. The interior surface 25 (the surface that faces the inside of the chamber) contains convex lenses 27 that run the entire horizontal length L of wall 22. In this way, while the light/dark regions change in terms of the vertical profile V of the flat panel PBR, the light/dark regions are consistent along the horizontal length L.

The change in light/dark regions along the vertical profile V is by design because the one or more spargers placed along the bottom of the chamber 20 will cause the bubbles to travel upwards, thereby causing a vertical current that causes the photosynthetic micro-organisms to move up and down the height of the chamber. As the micro-organisms travel vertically through the chamber 23, the micro-organisms will go through a multitude of light and dark regions. If the movement were to flow along another vector (e.g., horizontally), then the light/dark regions would typically alternate along the path of the vector and be consistent perpendicular to that vector.

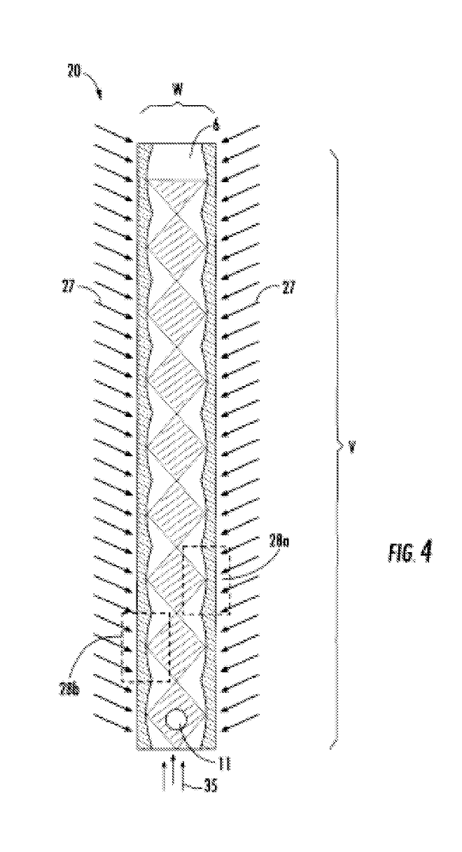

FIG. 4 provides another example of a lensed flat panel PBR constructed in accordance with the invention. More particularly, FIG. 4 provides a cross sectional side view a chamber 20 of a lensed flat panel PBR. FIG. 4 is identical to FIG. 2 with the exception that the interior walls 25a and 25b curve inward to form multiple concave lenses 28c and 28d. This produces essentially the same effect, with the exception that the alternating high light regions 31 and dark regions 33 align differently with respect to the curvature of the lenses.

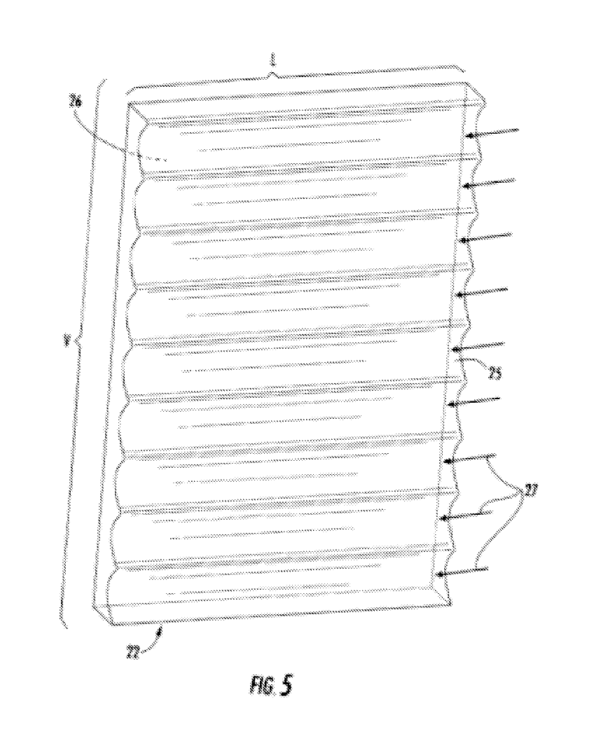

FIG. 5 further illustrates the lensed flat panel PBR shown in FIG. 4. More particularly, FIG. 5 shows a perspective view of one wall 22 in chamber 20 of FIG. 4. In FIG. 5, wall 22 has a length L and a height V. The outer surface 26 is substantially smooth and flat. The interior surface 25 (the surface that faces the inside of the chamber) contains concave lenses 27 that run the entire horizontal length L of wall 22. In this way, again, the light/dark regions change in terms of the vertical profile V of the flat panel PBR, the light/dark regions are consistent along the horizontal length L.

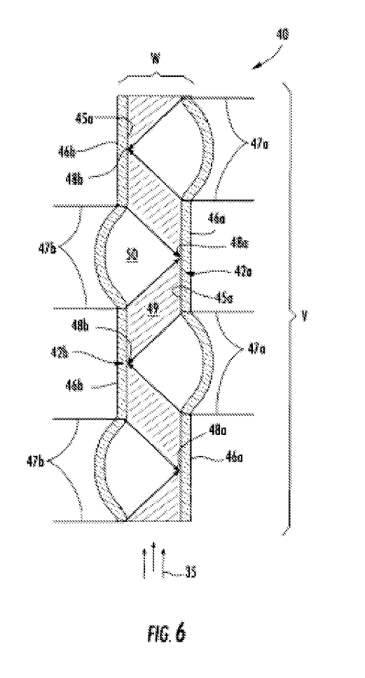

Another example of a lensed flat panel PBR is illustrated by FIG. 6. FIG. 6 provides a cross sectional side view of another chamber 40 of a lensed flat panel PBR.

In FIG. 6, front wall 42a and back wall 42b, respectively, have negative meniscus lens regions 47a and 47b. The walls 42a and 42b additionally have adjacent non-lensed (i.e., flat or substantially or predominantly flat) regions 46a and 46b. Each non-lensed region 46a and 46b is positioned between lensed regions 47a and 47b on the same wall and opposite a lensed region 47a and 47b on the opposite wall. In this configuration, approximately half of the surfaces of the walls 42a and 42b are formed into lensed regions 47a and 47b. In this configuration, lensed regions 47a and 47b are shaped such that light is focused at a point 48a and 48b at or near the interior surface 45a and 45b of the opposing wall. Incident light hits the exterior surface 46a and 46b of walls 42a and 42b and is focused by lensed regions 47a and 47b to a point 48a and 48b at or near the interior surface of the opposing wall.

In the example of FIG. 6, the light/dark profile is different from the light/dark profile in the example of FIG. 2. In FIG. 6, the dark regions are represented by roughly serpentine trapezoidal regions 49 and the light regions are represented by spaced roughly triangular regions 50.

In FIG. 6, the exact pattern of light and dark regions show is based on the assumption that incidental light will approach the walls perpendicularly. However, as in FIGS. 2 and 4 light need not hit the chamber walls perpendicularly or at any particular angle to produce the desired effect. If the PBR is not designed to continuously move to face the sun, then the exact shape of the pattern of light and dark regions may vary slightly as the angle of approaching light from the sun varies at different times of the day.

In FIG. 6, the direction of the flow of the media in this portion of the chamber is indicated as flowing upward by arrows 35. The flow passes micro-organisms vertically through the alternating high light and dark regions.

The physical dimensions of a lensed flat panel PBR in terms of horizontal length and vertical height can vary significantly. In terms of width, a lensed flat panel PBR can be similar to conventional photobioreactors—but one of the advantages of the invention is that it also can be much wider. Wider widths are enabled by the fact that focused light can penetrate deeper into cultures of light absorbing micro-organisms than non-focused light. For example, the width of a lensed flat panel PBR may be 2 to 3 times thicker than traditional flat panel PBRs (e.g., anywhere from 5 to 15 cm and, preferably, from 10 to 15 cm). The exact width will depend on the light/dark profile desired which, in turn, will depend on the needs of the specific micro-organism being grown. Generally, a wider chamber that has more volume is desired in order to increase production per photobioreactor and, thereby, reduce capital and operating cost per unit volume of culture grown.

The ideal lens dimensions can be determined according to the Lensmaker's equation. For a plano-convex lens, as shown in FIG. 2, the focal length of the focused light, f, can be defined as 1/f=(nₘₐₜₑᵣᵢₐₗ/n_{media}−1) (1/R). Here nₘₐₜₑᵣᵢₐₗ is the refractive index of the plastic or glass wall material, n_{media} the refractive index of the media containing the micro-organism, and R is the radius of the curvature of the lens. For example, if the width of the flat panel PBR is 5 cm, and if one desires to focus the light to the center of the PBR (i.e., f=2.5 cm), and if nₘₐₜₑᵣᵢₐₗ equals 1.5 and n_{media} equals 1.33, then the resulting radius for the curvature of the lens R should be about 0.32 cm. However, the lens curvature need not be determined by any specific equation.

The curvature of the lenses employed will depend on the width of the chamber. Typically, the focal length of each lens is identical. Preferably, the focal length of each lens is selected from a value ranging from approximately ¼ the chamber width to approximately the entire chamber width. Thus, if nₘₐₜₑᵣᵢₐₗ equals 1.5 and n_{media} equals 1.33, and the width of the chamber with a plano-convex lens equals 5 cm, then the radius of the curvature for each lens will typically range from about 0.15 cm to about 0.65 cm. Alternatively, if the width of the chamber equals 15 cm, then the radius of curvature for the convex lens will typically range from about 0.5 cm to about 1.9 cm. More preferably, the focal length of each lens is selected from a value ranging from approximately ½ the chamber width to approximately the entire chamber width.

The examples shown in FIGS. 2-6 illustrative only three of many possible configurations for lensed PBRs generally, and lensed flat panel PBRs specifically, that are embraced by the present invention. Many other alternative configurations are possible. The exact configuration will be dictated by the type of PBR utilized, the means for providing directional current in the PBR, and the desired light/dark profile for the micro-organism being grown. Variations in configuration can include the type, number, position and curvature of the lenses and the width of the photobioreactor. As stated, the lenses may be selected from any type including, but not limited to, biconvex, biconcave, plano-convex, plano-concave, meniscus (i.e., convex-concave) lenses, or any combination thereof, so long as the lenses focus or disperse light in a known manner. By varying the number, position, and shape of the lens, and the width of the photobioreactor, the number, shape and intensity of the light and dark regions can be controlled. Light regions are made brighter and dark regions darker than otherwise possible and micro-organisms are exposed to more frequent and regular alternating light/dark cycle than otherwise possible.

The ideal lensed PBR is a closed flat panel photobioreactor comprising a chamber for holding media, formed from a plurality of panels including a front and back panel, wherein the front and back panels are translucent or transparent and form multiple convex and/or convex lenses that provide a plurality of light and dark portions inside the chamber. Each lens converges light coming into the chamber at a focal distance ranging from about ¼ of the width of the chamber (and, preferably ½ the width of the chamber) to about the width of the chamber. The light and dark regions are substantially uniform and unchanging across the horizontal length of the chamber but alternate back and forth between light and dark regions across the vertical height of the chamber.

Although it is preferable to mold or etch the lenses as part of the manufacturing process when making the panel walls of the PBR chamber, the lenses can also be adhered to or otherwise attached to the panels. In fact, such devices need not be touching the panels at all so long as light entering the chamber is focused to create high light and low light (dark) areas within the chamber of the PBR.

As with conventional photobioreactors, multiple lensed PBRs can be serially connected to one another. In the case of flat panel photobioreactors, this has the benefit of diffusing light as it bounces between, or travels through, adjacent chambers. Some micro-organisms, such as algae, grow significantly better in diffuse light as opposed to direct light.

### Striped PBR

As an alternative to lensed panels or in addition to lensed panels, the invention includes chambers wherein at least one panel of the chamber comprises a pattern of alternating transparent or translucent regions and opaque regions to generate a plurality of light and dark regions within the chamber. Relatively lighter regions will be created adjacent to the transparent or translucent regions. Relatively darker regions will be created adjacent to the opaque regions. A convenient pattern for the opaque regions is a uniformly spaced striping and, more preferably, a uniformly spaced striping across the horizontal length of the chamber. However, the pattern of opaque regions can be any shape, width and frequency in order to achieve the light/dark profile desired. For the purposes of this description, this arrangement will be called a “striped PBR.”

As with the lensed PBR, the chamber walls of a striped PBR are typically made of smooth rigid glass or plastic (for example acrylic). The pattern of opaque material can be formed on any one or a multiple of walls during manufacture by incorporating opaque dyes, coatings or film. This is preferred technique of forming the pattern from a structural integrity standpoint. Alternatively, or in addition, the opaque pattern may be provided post wall manufacture by adhering an opaque coating or film (e.g., tape). This is preferable from a cost standpoint.

The opaque material blocks some or all wavelengths of incoming light. Preferably the material blocks actinic light. More preferably, the material blocks ultraviolet light. Ideally, the material blocks all light.

The opaque material may be reflective. Reflective materials reduce heat absorption and maximize photosynthetic efficiency by utilizing reflected light elsewhere (e.g., in adjacent chambers). Preferably, the material reflects actinic light. More preferably the material reflects ultraviolet light. Ideally, the material reflects all light.

Any of the chamber walls may incorporate the pattern of opaque material, but is preferably on opposing walls. Preferably, but not necessarily, the patterns on the opposite walls directly align with one another.

Further, the pattern of opaque material can be positioned on the interior and/or exterior wall surfaces. However, if the opaque pattern is generated by an adhered material, then the material is preferably only located on exterior wall surfaces to prevent fouling. The exact configuration of the opaque pattern depends on the specific light/dark profile desired. This, in turn, depends on the type of photobioreactor utilized (e.g., tubular or flat panel) and the optimal light/dark profile requirement of the particular species of micro-organism being grown.

Preferably, the striped PBR is a striped flat panel photobioreactor (hereinafter “striped flat panel PBR”). Even more preferably, the striped flat panel PBR has a chamber comprising opaque patterns on its front and/or back panels (i.e., the panels having the largest surface area). For example, the opaque patterns may be positioned in spaced parallel rows to create alternating rows of light exposure within the chamber, wherein rows of light regions are interspaced with rows of dark regions, each extending parallel to the chambers horizontal axis (i.e., length). Thus, light is substantially uniform across a majority, if not all, horizontal cross-sections taken parallel to the length of the chamber but alternates along a majority, if not all, vertical cross-sections taken parallel to the height of the chamber. This creates a vertical light/dark profile that consistently alternates between light and dark across the majority, and preferably the entire, vertical axis (height) of the photobioreactor and a horizontal light/dark profile that remains substantially uniform (i.e., stays consistently light or consistently dark) across the majority, and preferably the entire, horizontal length of the photobioreactor.

In a striped flat panel PBR, a vertically alternating light/dark profile is desired since micro-organisms typically flow up and down in the chamber roughly in-line with vertical currents created by one or more spargers at the bottom of the chamber. Flow within a striped flat panel PBR is typically generated by bubbling CO₂ and/or air through one or more spargers located at the bottom of the flat panel. As the bubbles rise, they generate liquid flow circulation within the chamber causing the micro-organisms in the media to flow up and down.

In a striped flat panel PBR, the frequency of the light/dark cycle can be optimized by varying the interplay between the opaque pattern and the bubbling rate of resource gases (e.g., air and CO₂). The bubbling rate changes the velocity of the fluid which, in turn, changes the frequency of the light/dark cycle experienced by the micro-organisms. Preferred configurations, therefore, include a striped flat panel PBR wherein the interplay between the opaque pattern (i.e., stripes) and the bubbling rate produced by one or more spargers at or near the bottom of the chamber combine to expose micro-organisms in the chamber to a light/dark cycle that alternates at a frequency ranging from 25 microseconds to 60 seconds. Preferably, the light/dark cycle alternates at a frequency ranging from 1 millisecond to 60 seconds. Ideally, the alternating light/dark cycle alternates at a frequency ranging from 1 second to 10 seconds.

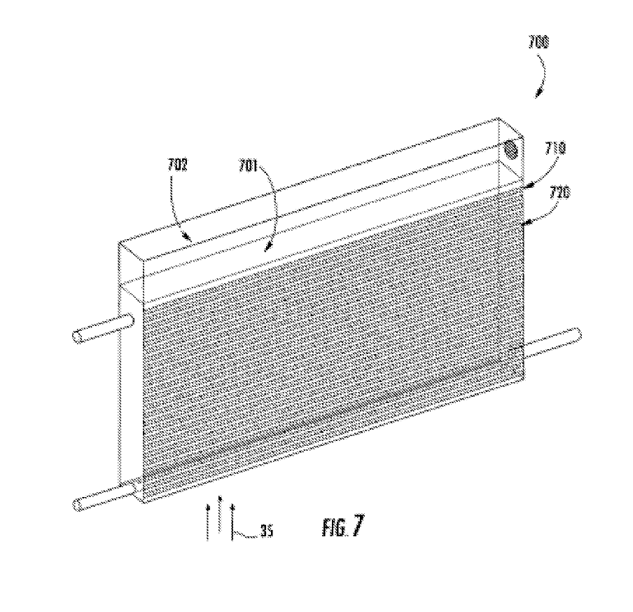

One example of a striped flat panel PBR is illustrated by FIG. 7. In FIG. 7, the photobioreactor comprises a chamber 700. An otherwise transparent or translucent front wall 701 of chamber 700 is covered with multiple rows of reflective film 710 positioned parallel to its horizontal axis (length). The rows of reflective film 710 are uniformly spaced and sufficiently thick to cover approximately half of the surface area of front wall 701. In between each row of reflective film 710 is an uncovered row 720 that remains transparent or translucent. Preferably, back wall 702 of chamber 700 is also transparent or translucent and contains an identical pattern of reflective film. Incident light hits front wall 701 and/or back wall 702 and is reflected away from chamber 700 and, preferably, toward any adjacent chambers of other photobioreactors (not shown). In this way, the interior of chamber 700 contains an alternating pattern of light regions and dark regions. If light approaches perpendicular to the chamber 700, then the light regions will be immediately behind the uncovered rows 720 and the dark regions will be immediately behind the reflective rows 710. However, light need not hit chamber 700 perpendicularly or at any particular angle to produce the desired effect. If the striped photobioreactor is not designed to continuously move perpendicular to the sun, then the exact shape of the pattern of light and dark regions may vary slightly as the angle of approaching light from the sun varies at different times of the day.

In FIG. 7, the direction of the flow of the media in this portion of the chamber is indicated as flowing upward by arrows 35. The flow passes micro-organisms vertically through the alternating high light and dark regions.

The example shown in FIG. 7 illustrates only one possible configuration for striped PBRs generally, and striped flat panel PBRs specifically, embraced by the present invention. Many other alternative configurations are possible. The exact configuration will be dictated by the type of PBR utilized, the means for providing directional current in the PBR, and the desired light/dark profile for the micro-organism being grown. Variations in configuration can include the dimensions, number, position and concentration of regions of opaque material. By varying the dimensions, number, position, and concentration of regions of opaque material, the number and shape of the light and dark regions can be controlled and micro-organisms are exposed to more frequent and regular alternating light/dark cycle than otherwise possible.

The ideal striped PBR is a closed flat panel photobioreactor comprising a chamber for holding media, formed from a plurality of panels including a front and back panel, wherein the front and/or back panels are translucent or transparent and contain a repeating pattern of spaced opaque material. The spaced opaque material covers at least 40% of the surface area of a panel (and preferably approximately 50% of the surface area). The light and dark regions are substantially uniform and unchanging across the horizontal length of the chamber but alternate back and forth between light and dark regions across the vertical height of the chamber.

As with conventional photobioreactors, multiple striped PBRs can be serially connected to one another. In the case of flat panel photobioreactors, this has the benefit of diffusing light as it bounces between, or travels through, adjacent chambers. As stated, some micro-organisms, such as algae, grow significantly better in diffuse light as opposed to direct light.

### Hybrids

In will be understood that the invention also encompasses photobioreactors that combine the above described features of the lensed PBR and the striped PBR. Accordingly, PBRs that use a combination of lenses and stripes to generate light and dark regions are embraced.

### Methods

The invention also pertains to a method for increasing the biomass and/or bioproduct productivity of photosynthetic micro-organisms (e.g., preferably algae) in a photobioreactor comprising at least two steps. The first step is redirecting or blocking at least a portion of light that reaches a surface of the photobioreactor to create a plurality of alternating light and dark regions within the photobioreactor. The second step is moving micro-organisms directionally through said light and dark regions. For the purposes of this method, the light is preferably incident sunlight as opposed to artificial illumination.

To redirect light, a lensed PBR as described above can be utilized. Accordingly, in one embodiment of the method, the light and dark regions are created using one or more lenses formed in, or adhered to, one or more walls of the photobioreactor. For the purposes of this aspect of the invention, all of the discussion above pertaining to lensed PBR is hereby incorporated.

To block light, a striped PBR as described above can be utilized. Accordingly, in another embodiment of the method, the light and dark regions are created by covering a portion of one or more walls of a photobioreactor with an opaque material. Ideally, the opaque material is reflective. For the purposes of this aspect of the invention, all of the discussion above pertaining to striped PBR is hereby incorporated.

In all embodiments of the method, the photobioreactor is preferably a flat panel photobioreactor. In such case, the light regions and dark regions alternate along the vertical height of the chamber.

### Sparger/Baffles

All of the apparatuses and methods described herein may additionally include or use baffles positioned in the photobioreactor to increase mixing. Baffles are designed to induce convective circulation within a photobioreactor. The number, position and size of the baffles can vary greatly depending on the type of photo-synthetic micro-organisms and the desired convective current.

However, a particular sparger/baffle design has been discovered to be particularly beneficial in generating alternating vertical current in flat panel bioreactors. Accordingly, the invention also pertains to a method for generating an alternating vertical current in a closed photobioreactor, for example a closed PBR as described herein, comprising a chamber for growing photosynthetic micro-organisms that comprises the steps of (1) installing one or more baffles in the chamber and (2) releasing gas (e.g., nutrient gas) into the bottom of the chamber at different rates on either side of each baffle.

More particularly, if baffles are used, then, preferably, the baffles are aligned so leaving space above and below each baffle for media to flow around the baffle. This, effectively, partitions the PBR chamber into subchambers—the borders of each subchamber being formed by a baffle and an immediately adjacent baffle or chamber wall. The flow-rate of the air/CO₂ sparging into each subchamber is then varied in an alternating manner. In other words, in one subchamber gas is released at a faster rate than in the immediately adjacent subchamber or subchambers. In this manner, a circular vertical current is created around each baffle such that media flows upward on one side and downward on the opposite side.

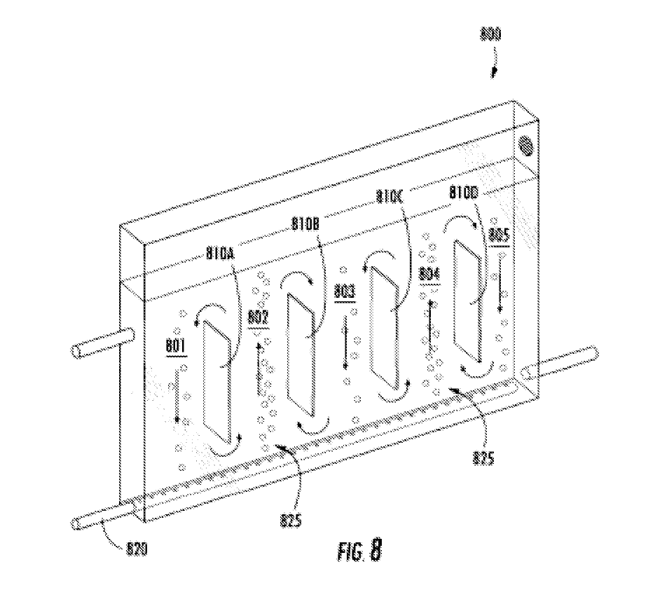

FIG. 8 is illustrative. In FIG. 8, four baffles 810 A-D divide a chamber 800 into five subchambers 801, 802, 803, 804 and 805. Each subchamber 801, 802, 803, 804 and 805 is defined by the area between each baffle and the immediately adjacent baffle or chamber wall. One or more spargers (820) are used to introduce current by bubbling gas (825) into each chamber. Gas is introduced at a relatively higher rate in subchambers 802 and 804 than in subchambers 801, 803 and 805, thereby forcing the flow of media upward in the subchambers 802 and 804. Conversely, gas is introduced at a relatively lower rate in subchambers 801, 803 and 805 than in subchambers 802 and 804, thereby forcing the flow of media downward in subchambers 801, 803 and 805. In such a case, the dominant current in each subchamber, indicated by arrows, flows in the opposite vertical direction as the immediately adjacent subchambers. In this manner, flow rises and falls at alternating points along the horizontal length of the PBR so that photosynthetic micro-organisms continuously flow up and down through alternating light and dark regions.

### Micro-organisms

All of the apparatuses and methods described herein can be used with any type of photosynthetic micro-organism—but work especially well with organisms, such as algae, that require light/dark cycle or intermittent light effect to achieve optimal growth. Examples of suitable algae include rhodophytes, chlorophytes, heterokontophytes, tribophytes, glaucophytes, chlorarachniophytes, euglenoids, haptophytes, cryptomonads, dinoflagellums, phytoplanktons, and the like, and any combination thereof. In one embodiment, the algae is selected from the classes Chlorophyceae and/or Haptophyta. Specific species for use in the invention can include, but are not limited to, Neochlorisoleoabundans, Scenedesmusdimorphus, Euglena gracilis, Phaeodactylumtricornutum, Pleurochrysiscarterae, Prymnesiumparvum, Tetraselmischui, and Chlamydomonasreinhardtii. Additional or alternate algal sources can include one or more microalgae of the Achnanthes, Amphiprora,Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Platymonas, Pleurochrysis, Pleurococcus, Prototheca, Pseudochlorella, Pyramimonas, Pyrobotrys, Scenedesmus, Skeletonema, Spyrogyra, Stichococcus, Tetraselmis, Thalassiosira, Viridiella, and Volvox species, and/or one or more cyanobacteria of the Agmenellum,Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species.

### Potential Advantages

The photobioreactors and methods described herein provide a number of advantages compared to traditional photobioreactors. Among other things, the apparatuses and methods described herein provide a relatively passive means for exposing photosynthetic micro-organisms to an effective light/dark cycle. In addition to creating more light and dark regions, the regions of light created tend to be lighter, and the regions of dark created tend to be darker than otherwise obtainable. Further, the overall area of light areas versus dark, in terms of percentage, can be precisely controlled and optimized to suit the specific needs of specific photosynthetic micro-organisms. In the invention, as the micro-organisms move or are made to move directionally through the photobioreactor, they experience alternating regions of relatively high and low light without significant energy input.

Additional advantages are obtained by using a lensed PBR in particular. Specifically, the width of the media chamber in a lensed PBR can be manufactured to be significantly thicker than is otherwise possible because light can be focused by the lenses to penetrate deeper into colonies of light absorbing micro-organisms. As a result, the volume of micro-organism containing media per photobioreactor can be increased, thereby making the photobioreactor more cost effective in terms of micro-organism growth. This reduces the number of photobioreactors are required to achieve the same productivity, resulting in significant savings in both capital and operational costs. Note that, in a lensed PBR, despite the fact that dark zones are created in the photobioreactor, the total light available in the photobioreactor does not change. Instead light is redirected and regions of more intense light are created.

Additional advantages are also obtained by using a reflective striped PBR in particular. Namely, a striped PBR that employs a reflective material reduces the heating experienced by the algae culture. In other words, if only a fraction of the incident sunlight is allowed to enter the photobioreactor, while the remainder is reflected elsewhere, then each photobioreactor does not heat up as intensely. This, in turn, reduces the need for, and thereby the cost of, cooling—which is often accomplished by spraying water on the device. If the reflected light is reflected in the direction of adjacent photobioreactors, this also improves total incident light utilization.

It should be understood that there can be various modifications, adjustments and applications of the disclosed invention that would be apparent to those of skill in the art, and the present application is intended to cover any such embodiments. For example, while the invention has been described in terms of alternative uses of lenses or opaque material to generate a light/dark cycle, it should be readily apparent, and is certainly herein embraced, that combinations of these separate techniques can be employed in a single photobioreactor and, further, that photobioreactor arrays using both types of photobioreactors can be employed. In addition, any and all the techniques described herein, and any combination thereof, can be utilized in combination with other light/dark cycle inducing measures known in the art, such as vigorous mixing. Accordingly, while the present invention has been described in the context of certain preferred embodiments, it is intended that the full scope of the invention be measured by reference to the scope of the following claims.

What is claimed is:

1. A closed photobioreactor for growing photosynthetic micro-organisms comprising a chamber for holding media formed from a plurality of panels, wherein at least one panel contains translucent or transparent portions and, further, redirects or absorbs light to provide a plurality of alternating light and dark regions within the chamber, wherein the chamber is made of one or more translucent or transparent panels that form or comprise one or more convex and/or concave lenses that converge and/or diverge light to generate a plurality of alternating light and dark regions within the chamber, and wherein the interplay between radius of the curvature of the lenses and bubbling rate produced by one or more spargers located at or near the bottom of the chamber combine to expose micro-organisms in the chamber to a light/dark cycle that alternates at a frequency ranging from 25 microseconds to 60 seconds.

2. The photobioreactor of claim 1, wherein the light/dark cycle alternates at a frequency ranging from 1 millisecond to 60 seconds.

3. The photobioreactor of claim 1, wherein the light/dark cycle alternates at a frequency ranging from 1 second to 10 seconds.

4. The photobioreactor of claim 1, wherein the photobioreactor is a flat panel photobioreactor and wherein the chamber contains a plurality of light and dark regions that each remain substantially uniform across the chamber's horizontal length but alternate between light and dark regions across the chamber's vertical height.

5. The photobioreactor of claim 1, wherein the lenses are selected from the types consisting of biconvex, biconcave, plano-convex, plano-concave, meniscus lenses and any combination thereof.

6. The photobioreactor of claim 1, wherein the lenses are at least formed on the interior surface of a translucent or transparent panel.

7. The photobioreactor of claim 1, wherein the focal length of the lenses range from approximately ¼ the internal width of the chamber to approximately the entire internal width of the chamber.

8. The photobioreactor of claim 1, wherein chamber further comprises at least one opaque region, such that the chamber comprises a pattern of alternating transparent or translucent regions and opaque regions to generate a plurality of light and dark regions within the chamber.

9. The photobioreactor of claim 8, wherein the opaque region(s) is(are) made by forming and/or adhering an opaque material to the exterior surface of one or more panels.

10. The photobioreactor of claim 9, where the opaque material is reflective.

11. A method for increasing the growth rate and growth density of photosynthetic micro-organisms in the photobioreactor according to claim 1 comprising at least the following steps:
(a) redirecting or blocking at least a portion of the light that reaches a surface of the photobioreactor to create a plurality of alternating light and dark regions within the photobioreactor; and
(b) moving micro-organisms directionally through said light and dark regions.

12. The method of claim 11, wherein the micro-organism comprises algae.

13. The method of claim 11, wherein the light and dark regions are created using the one or more lenses formed in, or adhered to, one or more walls of the photobioreactor.

14. The method of claim 11, wherein the light and dark regions are created by covering a portion of one or more walls of the photobioreactor with an opaque material.

15. The method of claim 11, wherein the plurality of alternating light regions and dark regions alternate along the vertical axis of a chamber in the photobioreactor.

16. The method of claim 15, wherein the plurality of alternating light regions and dark regions further remain substantially uniform across the chamber's horizontal length.

17. The method of claim 15, wherein one or more baffles are positioned in the chamber and nutrient gas is released into the bottom of the chamber at different rates on either side of each baffle.

18. A method for generating an alternating vertical current in a closed photobioreactor comprising a chamber for growing photosynthetic micro-organisms, wherein said method comprises the following steps:
(a) positioning one or more baffles in the chamber using the method according to claim 15; and
(b) releasing nutrient gas into the bottom of the chamber at different rates on either side of each baffle.
